(19) United States
(12) Patent Application Publication
(10) Pub. No.: US 2026/0215971 A1
(43) Pub. Date: Jul. 30, 2026

(54) APPARATUS AND METHOD FOR ABSORBING NASAL SECRETIONS DURING MAKEUP APPLICATION

(71) Applicant: Jennifer Lynn Burke, Rocky Hill, CT (US)

(72) Inventor: Jennifer Lynn Burke, Rocky Hill, CT (US)

(21) Appl. No.: 19/083,033

(22) Filed: Mar. 18, 2025

Related U.S. Application Data
(60) Provisional application No. 63/751,568, filed on Jan. 30, 2025.

(51) Int. Cl. A61F 13/12

(52) U.S. Cl. CPC A61F 13/126

(57) ABSTRACT

An apparatus and method for absorbing nasal secretions during makeup application or daily activities is disclosed. The invention features an absorbing nose plug assembly comprising a pair of absorbent nose plugs made from materials such as cotton or bamboo, connected by a septum tab. The septum tab, made from an earth-friendly material, is designed with an inward bend to secure the plugs within the nostrils, effectively absorbing nasal secretions. This solution offers a convenient and hygienic way to manage nasal secretions without disrupting makeup application. The assembly may include features like a tapered shape for comfort and antimicrobial properties.

### CROSS-REFERENCE TO RELATED APPLICATION

This application claims the benefit of priority of U.S. provisional application number 63/751,568, filed January 30, 2025, the contents of which are herein incorporated by reference.

### BACKGROUND OF THE INVENTION

When applying makeup, many individuals encounter the challenge of nasal secretions, which can disrupt the application process and affect the final appearance. This issue is not uncommon, as evidenced by numerous makeup enthusiasts and influencers who have shared similar experiences. The problem is exacerbated by the lack of effective solutions specifically designed to address nasal secretions during makeup application.

Currently, the only workaround involves using larger swabs, which are cumbersome and inconvenient due to their protruding sticks. This method is not only awkward but also ineffective, as the method can interfere with the makeup process. The lack of a dedicated, efficient solution highlights the need for an innovative approach to manage nasal secretions seamlessly during makeup application and other daily activities.

### SUMMARY OF THE INVENTION

In one aspect of the present invention, there is disclosed an absorbing nose plug assembly for absorbing nasal secretions during makeup application or daily activities, comprising a pair of absorbent nose plugs formed of a material selected from the group consisting of cotton and bamboo; and a septum tab that connects the pair of absorbent nose plugs, the septum tab comprising an earth-friendly material and configured with an inwardly bent structure for insertion into a user’s nostrils to secure the nose plugs in place; wherein the septum tab and absorbent nose plugs cooperate to position the absorbent material substantially within the nostrils to absorb nasal secretions.

In another embodiment, the absorbing nose plug assembly is further characterized by a tapered shape to accommodate various nostril sizes, enhancing fit and comfort. The absorbent nose plugs may be made from a material selected from the group consisting of hemp and flax, providing additional biodegradable options. The septum tab may include a pull tab to facilitate easy insertion and removal from the nostrils and may be made from recycled materials, offering an alternative to bamboo while maintaining earth-friendly characteristics. The septum tab may be configured with a specific inward bend angle preferably ranging from 15 to 30 degrees to optimize fit and comfort. Angles outside this range are contemplated and within the scope of the present invention. The absorbent nose plugs may include antimicrobial properties to enhance hygiene during use. The septum tab and absorbent nose plugs may be designed with a locking mechanism to ensure secure placement within the nostrils.

In a third embodiment, the disclosure includes a method for absorbing nasal secretions during makeup application or daily activities, comprising providing an absorbing nose plug assembly comprising a pair of absorbent nose plugs formed of a material selected from the group consisting of cotton and bamboo, and a septum tab connecting the pair of absorbent nose plugs, the septum tab comprising an earth-friendly material and configured with an inwardly bent structure for insertion into a user’s nostrils to secure the nose plugs in place.

These and other features will be more clearly understood from the following detailed description taken in conjunction with the accompanying drawings and claims.

### BRIEF DESCRIPTION OF THE DRAWINGS

FIG. 1 is a perspective view of an absorbing nose plug assembly constructed in accordance with a preferred embodiment of the present invention;

FIG. 2 is a front view of the absorbing nose plug assembly;

FIG. 3 is a front view of the absorbing nose plug assembly, shown in use;

FIG. 4 is a perspective view of an alternative embodiment of an absorbing nose plug assembly of the present invention;

FIG. 5 is a front view of the absorbing nose plug assembly of FIG. 4; and

FIG. 6 is a front view of the absorbing nose plug assembly of FIG. 4, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

When applying makeup, many individuals face the challenge of managing nasal secretions, which can disrupt the application process and affect the final appearance. This issue is prevalent among makeup enthusiasts and influencers, who often share their experiences on social media platforms. The problem is further compounded by the lack of effective solutions specifically designed to address nasal secretions during makeup applications. As a result, individuals are left to improvise with inadequate methods that fail to provide a satisfactory resolution.

Currently, the only workaround involves using larger cotton swabs, which are cumbersome and inconvenient due to their protruding sticks. This method is not only awkward but also ineffective, as the sticks can interfere with the makeup process, bumping into various surfaces and causing discomfort. The lack of a dedicated, efficient solution highlights the need for an innovative approach to manage nasal secretions seamlessly during makeup application and other daily activities.

The present invention addresses these and other challenges with conventional devices and methods by introducing an absorbing nose plug assembly designed specifically for absorbing nasal secretions during makeup application or daily activities. The assembly comprises a pair of absorbent nose plugs formed from materials such as cotton or bamboo, connected by a septum tab. The septum tab, made from an earth-friendly material, is configured with an inwardly bent structure for insertion into a user’s nostrils, securing the nose plugs in place. This design ensures that the absorbent material is positioned substantially within the nostrils, effectively absorbing nasal secretions without disrupting the makeup application process. The assembly of the present invention offers a convenient and hygienic solution, enhancing the overall experience for users.

The absorbing nose plug assembly of the present invention provides a practical solution for managing nasal secretions during makeup application or daily activities. By utilizing a pair of absorbent nose plugs formed from materials such as cotton or bamboo, the assembly ensures effective absorption of nasal secretions. The inclusion of a septum tab, made from an earth-friendly material, offers a secure and comfortable fit by being configured with an inwardly bent structure for insertion into the user's nostrils. This design not only positions the absorbent material substantially within the nostrils but also prevents interference with makeup application, unlike traditional methods involving larger swabs. The cooperation between the septum tab and the absorbent nose plugs enhances the assembly's ability to remain in place, providing a seamless and hygienic experience for the user. This arrangement addresses the shortcomings of existing solutions by offering a dedicated, efficient, and environmentally conscious approach to managing nasal secretions.

In various embodiments of the absorbing nose plug assembly, the septum tab can be constructed from a range of earth-friendly materials, such as bamboo, recycled plastics, or biodegradable polymers, each offering distinct environmental benefits and cost considerations. The absorbent nose plugs may be formed from materials like cotton, bamboo, hemp, or flax, providing options that cater to different levels of absorbency and biodegradability. The assembly can be designed with varying sizes and shapes of nose plugs, including tapered or bulbous configurations, to accommodate diverse nostril sizes and enhance user comfort. Additionally, the septum tab may feature different structural designs, such as a flexible or rigid form, with an inward bend angle that can be adjusted preferably between 15 to 30 degrees to optimize fit and comfort. Of course, angles outside this range are contemplated as a matter of design choice without departing from the spirit of the present invention.

The inclusion of a pull tab on the septum tab can facilitate easy insertion and removal, while the nose plugs may incorporate antimicrobial properties to improve hygiene during use. Furthermore, the assembly can be equipped with a locking mechanism to ensure secure placement within the nostrils, preventing accidental dislodgement during activities. These embodiments demonstrate the adaptability and versatility of the design within the scope of the patent claims, offering users a customizable and environmentally conscious solution for managing nasal secretions during makeup application or daily activities.

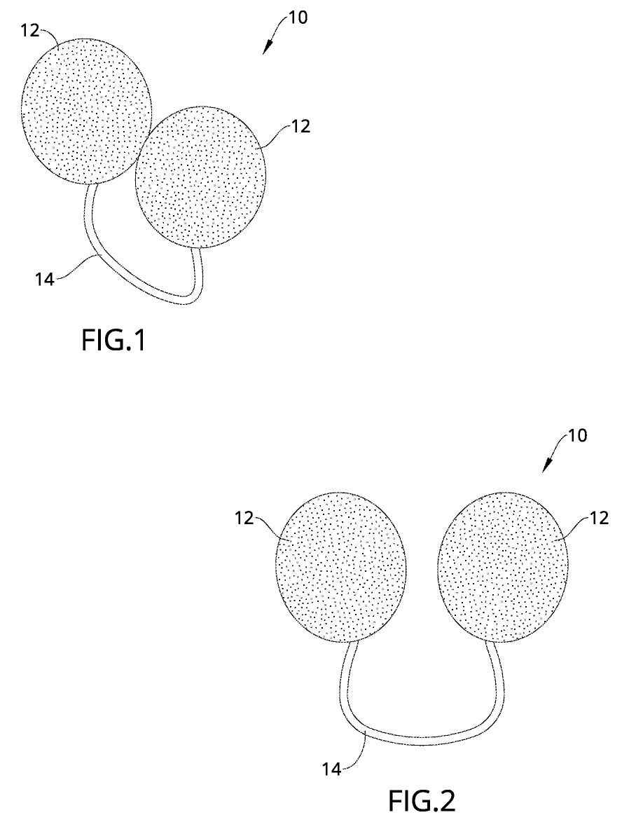

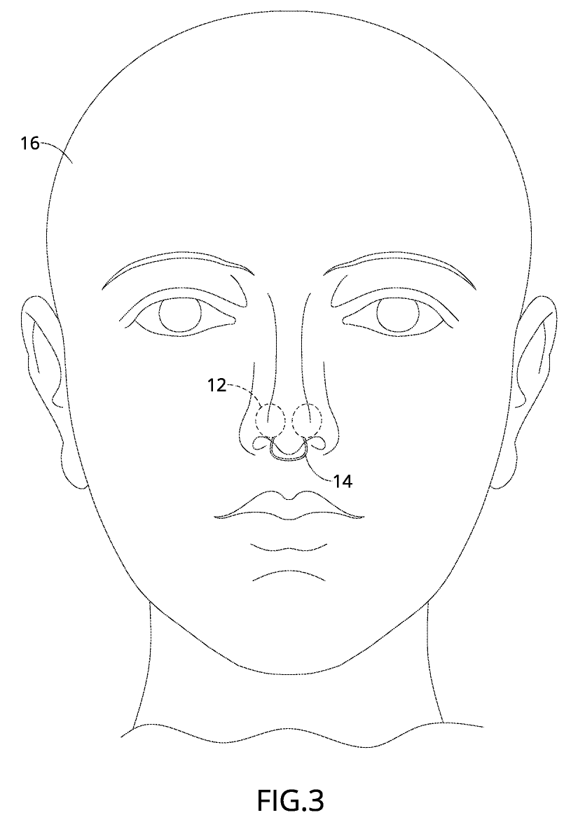

The nose plug assembly 10/20 of the present invention addresses the issue of nasal secretions during makeup applications or daily activities. As seen in FIGS. 1-6, the assembly 10/20 comprises a pair of absorbing nose plugs 12/22, which are connected by a septum tab 14/24. The nose plug assembly is engineered to be inserted into the nostrils of a user 16, providing a secure and comfortable fit that effectively absorbs nasal secretions. The design of the assembly ensures that the absorbent material is positioned substantially within the nostrils, thereby preventing any interference with makeup application. The assembly is intended to be a single-use, disposable solution, emphasizing its earth-friendly characteristics by utilizing biodegradable materials.

The absorbing nose plugs 12 play a significant role in the functionality of the nose plug assembly 10. These nose plugs 12 are formed from materials such as cotton or bamboo, chosen for their absorbent properties and biodegradability. The absorbing nose plugs 12 are designed to fit comfortably within the nostrils, providing an effective barrier against nasal secretions. The choice of materials allows for variations in absorbency and environmental impact, with options like hemp and flax also being viable alternatives. The design of the absorbing nose plugs 12 may include a tapered shape to accommodate various nostril sizes, enhancing both fit and comfort. Additionally, the nose plugs 12 can be treated with antimicrobial properties to improve hygiene during use.

The septum tab 14 functions as the connecting element between the pair of absorbing nose plugs 12 within the nose plug assembly 10. This component is crafted from an earth-friendly material, such as bamboo or recycled plastics, which contributes to the sustainability of the product. The septum tab 14 is configured with an inwardly bent structure, allowing insertion into the user's nostrils and securing the nose plugs 12 in place. The inward bend angle of the septum tab 14 can be adjusted preferably to between 15 to 30 degrees to optimize fit and comfort. Other angles are also within the scope of the present invention as a matter of design choice. In some embodiments, the septum tab 14 may include a pull tab 26 to facilitate easy insertion and removal, further enhancing user convenience. The design of the septum tab 14 ensures that the nose plug assembly 10 remains securely positioned within the nostrils, preventing accidental dislodgement during activities.

The user 16 is the individual who utilizes the nose plug assembly 10. The design of the assembly 10 is intended to provide a seamless and hygienic experience for the user 16, allowing them to manage nasal secretions effectively without disrupting their daily activities or makeup application. The user 16 benefits from the ergonomic design of the nose plug assembly 10, which is tailored to fit comfortably within the nostrils while remaining unobtrusive. The use of biodegradable materials in the construction of the assembly 10 aligns with the growing demand for environmentally conscious products, offering the user 16 a sustainable solution for managing nasal secretions.

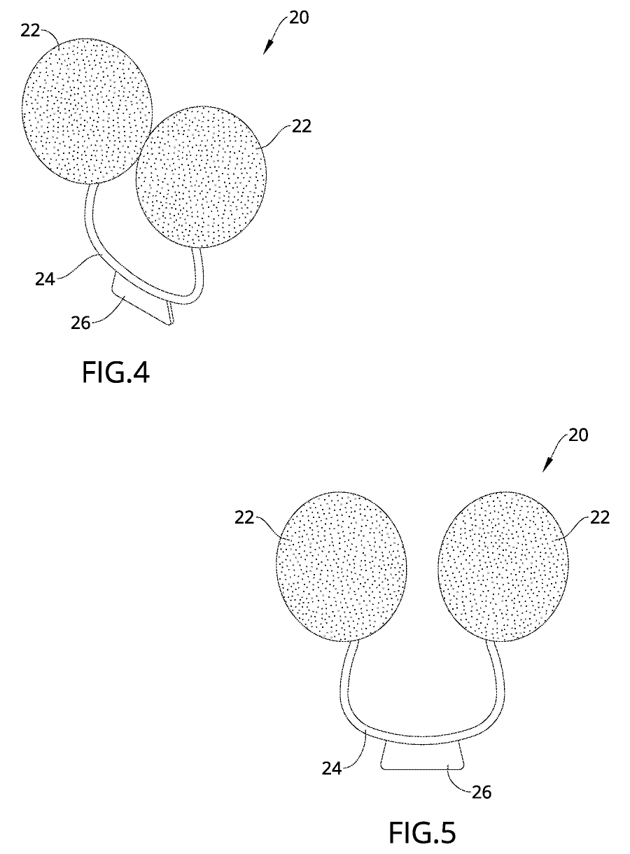

The nose plug assembly 20, as depicted in FIGS. 4-6, represents an alternative embodiment of the present invention. The nose plug assembly 20 comprises a pair of absorbing nose plugs 22, which are connected by a septum tab 24. This assembly is engineered to be inserted into the nostrils of a user 16, providing a secure and comfortable fit that effectively absorbs nasal secretions. The design ensures that the absorbent material is positioned substantially within the nostrils, thereby preventing any interference with makeup applications. The assembly is intended to be a single-use, disposable solution, emphasizing its earth-friendly characteristics by utilizing biodegradable materials.

The absorbing nose plugs 22 are an important component of the nose plug assembly 20, designed to fit comfortably within the nostrils and provide an effective barrier against nasal secretions. These nose plugs 22 are formed from materials such as cotton or bamboo, chosen for their absorbent properties and biodegradability. The choice of materials allows for variations in absorbency and environmental impact, with options like hemp and flax also being viable alternatives. The design of the absorbing nose plugs 22 may include a tapered shape to accommodate various nostril sizes, enhancing both fit and comfort. Additionally, the nose plugs 22 can be treated with antimicrobial properties to improve hygiene during use.

The septum tab 24 functions as the connecting element between the pair of absorbing nose plugs 22 within the nose plug assembly 20. This component is crafted from an earth-friendly material, such as bamboo or recycled plastics, which contributes to the sustainability of the product. The septum tab 24 is configured with an inwardly bent structure, allowing insertion into the user's nostrils and securing the nose plugs 22 in place. The inward bend angle of the septum tab 24 can be adjusted preferably between 15 to 30 degrees to optimize fit and comfort. Other angles outside this range are contemplated and within the scope of the invention. In some embodiments, the septum tab 24 may include a pull tab 26 to facilitate easy insertion and removal, further enhancing user convenience. The design of the septum tab 24 ensures that the nose plug assembly 20 remains securely positioned within the nostrils, preventing accidental dislodgement during activities.

The pull tab 26 is an optional feature of the septum tab 24, designed to aid in the insertion and removal of the nose plug assembly 20. This pull tab 26 provides an additional level of convenience for the user, allowing for quick and easy handling of the assembly. The inclusion of the pull tab 26 does not compromise the earth-friendly nature of the product, as the pull tab can be made from the same biodegradable materials as the septum tab 24. The pull tab 26 is strategically positioned to ensure that the pull tab does not interfere with the secure placement of the nose plugs 22 within the nostrils, maintaining the overall effectiveness of the assembly.

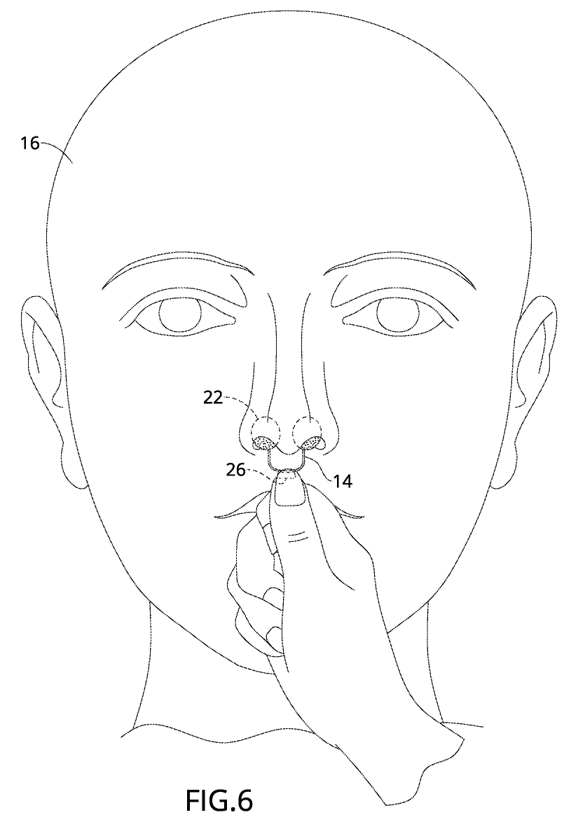

The user 16, as shown in FIG. 6, is the individual who utilizes the nose plug assembly 20. The design of the assembly 20 is intended to provide a seamless and hygienic experience for the user 16, allowing them to manage nasal secretions effectively without disrupting their daily activities or makeup application. The user 16 benefits from the ergonomic design of the nose plug assembly 20, which is tailored to fit comfortably within the nostrils while remaining unobtrusive. The use of biodegradable materials in the construction of the assembly 20 aligns with the growing demand for environmentally conscious products, offering the user 16 a sustainable solution for managing nasal secretions.

It should be understood, of course, that the foregoing relates to exemplary embodiments of the invention and that modifications may be made without departing from the spirit and scope of the invention as set forth in the following claims.

What is claimed is:

1. An absorbing nose plug assembly for absorbing nasal secretions during makeup application or daily activities, comprising:
a pair of absorbent nose plugs formed of a material selected from the group consisting of cotton and bamboo;
a septum tab that connects the pair of absorbent nose plugs, the septum tab comprising an earth-friendly material and configured with an inwardly bent structure for insertion into a user’s nostrils to secure the nose plugs in place;
wherein the septum tab and absorbent nose plugs cooperate to position the absorbent material substantially within the nostrils to absorb nasal secretions.

2. The absorbing nose plug assembly of claim 1, wherein the absorbent nose plugs are further characterized by a tapered shape to accommodate various nostril sizes, enhancing fit and comfort.

3. The absorbing nose plug assembly of claim 2, wherein the absorbent nose plugs are made from a material selected from the group consisting of hemp and flax, providing additional biodegradable options.

4. The absorbing nose plug assembly of claim 1, wherein the septum tab includes a pull tab to facilitate easy insertion and removal from the nostrils.

5. The absorbing nose plug assembly of claim 4, wherein the septum tab is made from recycled materials, offering an alternative to bamboo while maintaining earth-friendly characteristics.

6. The absorbing nose plug assembly of claim 1, wherein the septum tab is configured with a specific inward bend angle ranging from 15 to 30 degrees to optimize fit and comfort.

7. The absorbing nose plug assembly of claim 1, wherein the absorbent nose plugs include antimicrobial properties to enhance hygiene during use.

8. The absorbing nose plug assembly of claim 1, wherein the septum tab and absorbent nose plugs are designed with a locking mechanism to ensure secure placement within the nostrils.

9. A method for absorbing nasal secretions during makeup application or daily activities, comprising:
providing an absorbing nose plug assembly comprising:
a pair of absorbent nose plugs formed of a material selected from the group consisting of cotton and bamboo, and
a septum tab connecting the pair of absorbent nose plugs, the septum tab comprising an earth-friendly material and configured with an inwardly bent structure for insertion into a user’s nostrils to secure the nose plugs in place;
inserting the septum tab into the user’s nostrils such that the absorbent nose plugs are positioned substantially within the nostrils; and
absorbing nasal secretions during makeup application or daily activities.
